(19) United States
(12) Patent Application Publication
(10) Pub. No.: US 2006/0218688 A1
(43) Pub. Date: Oct. 5, 2006

(54) Eyeshade knitted structure made of bamboo charcoal fibers

(75) Inventor: Sen-Mei Cheng, Changhua Hsien (TW)

Correspondence Address: TROXELL LAW OFFICE PLLC;SUITE 1404, 5205 LEESBURG PIKE, FALLS CHURCH, VA, 22041, US

(73) Assignee: Taiwan Paiho Limited

(21) Appl. No.: 11/171,418

(22) Filed: Jul. 1, 2005

(30) Foreign Application Priority Data
Apr. 4, 2005 (TW) 094205184

(51) Int. Cl. A61F 9/00

(52) U.S. Cl. 2/12

(57) ABSTRACT

An eyeshade knitted structure made of bamboo charcoal fibers includes a multi-layer eyeshade made up of an intermediate breathable layer, a fine textural layer of resilient yarns and bamboo charcoal fibers knitted at one side of the breathable layer, and a decorative layer of resilient yarns and fibers knitted at the other side thereof wherein the breathable layer are interknitted with the fine textural layer and the decorative layer so that the eyeshade obtained thereby can be elastically adjusted according to the head size of a wearer to precisely fit to the skin around both eyes to achieve better light-blocking effect thereby. Besides, the eyeshade has a good ventilation structure to efficiently dissipate heat and eliminate sweat to avoid stuffy discomfort and maintain the hygiene of the eyeshade over long time of repeated application with an extra eye-care benefit via the fine textural layer having bamboo charcoal fibers knitted therein.

### BACKGROUND OF THE INVENTION

The present invention is related to an eyeshade knitted structure made of bamboo charcoal fibers, including a multi-layer eyeshade made up of an intermediate breathable layer interknitted with a decorative layer and a fine textural layer wherein the breathable layer has a plurality of airy pores densely disposed therein to boost the ventilation ability of the eyeshade and provide better upwards/downwards sustainability and rebound flexibility effect thereby so that the eyeshade thereof can precisely and comfortably fit to the skin around both eyes to achieve better light-blocking effect and efficient heat dissipation and sweat elimination ability to maintain the comfort ability of the eyeshade as well as to provide an extra eye-care benefit to the skin thereof via bamboo charcoal fibers of the fine textural layer knitted therein.

A conventional eyeshade on the market is usually applied to cover directly both eyes and block outside light thereby. Over long time of application onto the eyes, the conventional eyeshade featured by poor ventilation ability tends to cause stuffy discomfort to a wearer. In case of a sultry and hot weather, the eyeshade thereof stends to gather sweat and moisture around the skin of both eyes and cause humidity sticky and itchy feelings, which can easily result in the phenomena of allergy and irritation to the skin of eyes and endanger the health of the eyes covered in the eyeshade thereof. Thus, it's rather difficult to maintain the comfort ability of the conventional eyeshade in application thereof. Besides, the conventional eyeshade cannot properly fit to the skin around both eyes. Thus, outside light can be easily pervious through the eyeshade and influence its light block effect, which makes the eyes wearing it unable to get proper rest thereof.

Furthermore, the conventional eyeshade is not provided with the basic function such as light block effect and relaxing the strains of both eyes, while in present days the application of computers and the Internet becomes normal and widespread, people's eyes are easily afflicted with some uncomfortable problems, like lack of moisture, and fatigue which cannot be properly soothed by the conventional eyeshade. Thus, the conventional eyeshade's function is rather uncompetitive on the market.

### SUMMARY OF THE PRESENT INVENTION

It is, therefore, the primary purpose of the present invention to provide an eyeshade knitted structure made of bamboo charcoal fibers, including a multi-layer eyeshade made up of an intermediate breathable layer interknitted with a decorative layer and a fine textural layer both having resilient yarns knitted therein to provide better upwards/downwards sustainability and rebound flexibility so that the eyeshade acquired thereby can be synchronically stretched and flexibly adjusted according to the head size of a wearer to precisely and comfortably fit to the skin around both eyes thereof, avoiding light from outside to be pervious there-through to ensure proper rest of both eyes and achieve better light-blocking effect of the eyeshade thereof.

It is, therefore, the second purpose of the present invention to provide an eyeshade knitted structure made of bamboo charcoal fibers wherein the breathable layer is circularly knitted with the decorative layer and the fine textural layer there-between, and, via a plurality of airy pores densely arranged therein, the breathable layer can flexibly support and space apart the decorative layer and the fine textural layer to avoid muggy around there-between, efficiently achieving air-permeable, ventilating, and sweat-elimination effect thereby to boost the breathing ability of the eyeshade and to keep the eyes covered by it cool and comfortable so that the eyeshade can remain hygienically clean over long time of repeated application thereof.

It is, therefore, the third purpose of the present invention to provide an eyeshade knitted structure made of bamboo charcoal fibers wherein the fine textural layer having bamboo charcoal fibers knitted therein is at the inner side of the eyeshade in direct contact with the skin around both eyes thereof; and the bamboo charcoal fibers can emit far infrared radiation to soothe the nerves of eyes and blood pressure, improving blood circulation and relaxing the strains to properly fit to some people like computer groups, students, aged people, or some stressed office workers. Thus, the eyeshade is also provided with an extra eye-care benefit. Besides, the bamboo charcoal fibers thereof also equip negative ions to facilitate the activation of the cells thereby.

It is, therefore, the fourth purpose of the present invention to provide an eyeshade knitted structure made of bamboo charcoal fibers wherein the decorative layer, the fine textural layer, and the breathable layer of the eyeshade are synchronically interknitted via a circular knitting machine into a multi-layer form and integrally press-molded and tailor-made into a finished product, economically saving the processing time thereof to boost its competitive power on the market.

### BRIEF DESCRIPTION OF THE DRAWINGS

FIG. 1 is a diagram showing the application of the present invention.

FIG. 2 is a partially enlarged and cross sectional view of the present invention.

FIG. 3 is a diagram showing the present invention properly fit to the skin around both eyes with ventilating demonstration thereof.

FIG. 4 is a partially enlarged and cross sectional view of another embodiment of the present invention.

### DETAILED DESCRIPTION OF THE PREFERRED EMBODIMENTS

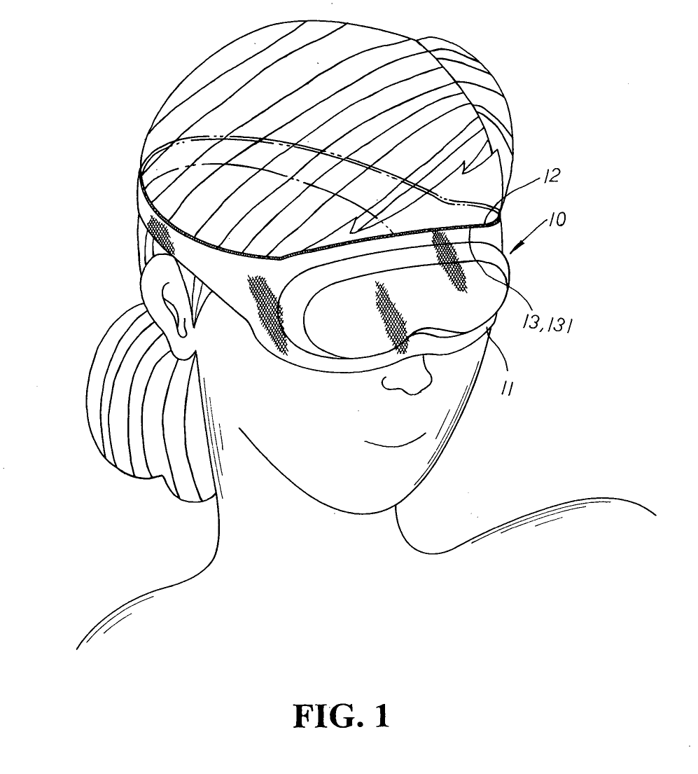

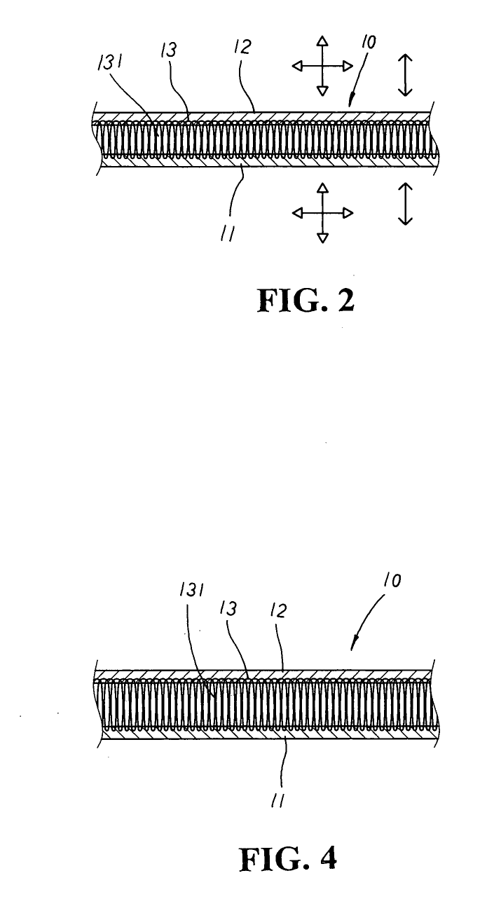

Please refer to FIG. 1. The present invention is related to an eyeshade knitted structure made of bamboo charcoal fibers, including an eyeshade 10 that, synchronically knitted via a circular knitting machine into a multi-layer form and integrally press-molded and tailor-made into a finished product, is made up of a decorative layer 11 of resilient yarns and fibers (of synthetic or natural materials, e.g. polyester) disposed at the outer side thereof, a fine textural layer 12 of resilient yarns and bamboo charcoal fibers disposed at the inner side, and an intermediate breathable layer 13 of pliable mono filaments reciprocally interknitted with the corresponding sides of the decorative layer 11 and the fine textural layer 12 thereof. The resilient yarns of the decorative layer 11 and the fine textural layer 12 thereof can be flexibly stretched and rebound. The breathable layer 13 thereof is interknitted with the decorative layer 11 and the fine textural layer 12 to form an intermediately interknitted cross section as shown in FIG. 2. A plurality of airy pores 131 among certain height and tied ring-shapes structure are densely arranged at the breathable layer 13 therein to correspondingly support the decorative layer 11 and the fine textural layer 12 thereof with a space formed there-between so that the airy pores 131 of the breathable layer 13 (referring to FIG. 3) promote to achieve sweat-elimination and ventilation effects thereby, and efficiently improve the air permeable ability of the eyeshade 10 so as to keep the eyes covered by it cool and comfortable and ensure the eyeshade 10 hygienically clean over long time of repeated application thereof. Furthermore, the decorative layer 11 and the fine textural layer 12, both having the resilient yarns knitted therein, are reciprocally interknitted with and softly supported by the intermediate breathable layer 13 thereof to equip better upwards/downwards sustainability and rebound flexibility thereof as shown in the double arrows of FIG. 2 so that the eyeshade 10 can be synchronically stretched and flexibly adjusted according to the head size of a wearer as shown in the cross arrows of FIG. 2. Thus, the adjusted eyeshade 10 can be precisely and comfortably fit to the skin around both eyes to avoid light outside pervious there-through, ensure proper rest of the eyes without being disturbed by outside light, and efficiently achieve better light-blocking and air permeable abilities of the eyeshade 10 thereof.

Furthermore, the fine textural layer 12 thereof is knitted at the inner side of the eyeshade 10 in direct contact with the skin around both eyes covered in the eyeshade 10, and the bamboo charcoal fibers knitted at the fine textural layer 12 therein can emit directly to human body far infrared radiation that can soothe the nerves of eyes and blood pressure, and improve better blood circulation. And, in addition to better light-blocking and air-breathing abilities thereof, the eyeshade 10 can also relax the strains of eyes on long time using, so it is especially suited for some people like computer groups, students, aged people, or some stressed office workers. Thus, the eyeshade 10 thereof is provided with an extra eye-care benefit thereof. Besides, the bamboo charcoal fibers thereof also equip negative ions to improve the activation of the cells thereby.

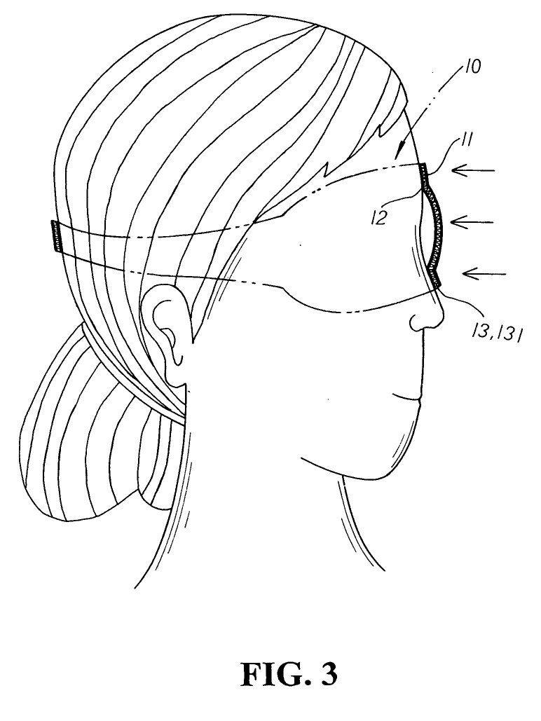

Please refer to FIG. 4 showing a partially enlarged and cross sectional view of another embodiment of the present invention. The eyeshade 10 can also have a breathable layer 13 whose height can be predetermined according to the setting of the circular knitting machine so as to knit the breathable layer 13 into various thickness as shown in FIGS. 2, 4 respectively with upwards/downwards sustainability and rebound flexibility structure thereof.

Besides, the eyeshade 10 are press-molded and tailor-made outwards into a curvature at a position relative to that of both eyes to form a proper distance there-between while accurately fitted to the skin around both eyes thereof.

What is claimed is:

1. An eyeshade knitted structure made of bamboo charcoal fibers, including a multi-layer eyeshade made up of an intermediate breathable layer, a fine textural layer of resilient yarns and bamboo charcoal fibers knitted at one side of the breathable layer, and a decorative layer of resilient yarns and fibers knitted at the other side of the breathable layer thereof wherein the breathable layer are reciprocally interknitted with both the fine textural layer and the decorative layer thereof so that the eyeshade acquired thereby can be elastically adjusted according to the head size of a wearer to comfortably and precisely fit to the skin around both eyes thereby; besides, the eyeshade also equips a good ventilation structure to efficiently dissipate heat and eliminate sweat as well as with an extra eye-care benefit to improve the blood circulation of the wearer thereby.

2. The eyeshade knitted structure made of bamboo charcoal fibers as claimed in claim 1 wherein the decorative layer, the fine textural layer, and the breathable layer of the eyeshade are synchronically interknitted via a circular knitting machine into a multi-layer form and integrally press-molded and tailor-made into a finished product.

3. The eyeshade knitted structure made of bamboo charcoal fibers as claimed in claim 1 wherein the breathable layer of the eyeshade is circularly knitted from mono filaments to provide better upward/downwards sustainability and rebound ability as well as softly flexibility and supporting effect thereby.

4. The eyeshade knitted structure made of bamboo charcoal fibers as claimed in claim 1 wherein the resilient yarns of the fine textural layer and the decorative layer thereof can be flexibly stretched and rebound in application thereof.

5. The eyeshade knitted structure made of bamboo charcoal fibers as claimed in claim 1 wherein the bamboo charcoal fibers of the fine textural layer thereof are made from bamboo charcoal yarns with an far infrared radiation effect provided therewith.

6. The eyeshade knitted structure made of bamboo charcoal fibers as claimed in claim 1 wherein the fibers of the decorative layer thereof can be made of natural or synthetic materials.

7. The eyeshade knitted structure made of bamboo charcoal fibers as claimed in claim 1 wherein the fine textural layer is knitted at the inner side of the eyeshade, and the decorative layer thereof is knitted at the outer side of the eyeshade thereof.

8. The eyeshade knitted structure made of bamboo charcoal fibers as claimed in claim 1 wherein the breathable layer thereof is circularly knitted with a form of certain height and tied ring-shapes which have airy pores densely disposed therein to flexibly support and separate both the decorative layer and the fine textural layer there-between.

9. The eyeshade knitted structure made of bamboo charcoal fibers as claimed in claim 1 wherein the height of the breathable layer of the eyeshade thereof can be predetermined according to the setting of the circular knitting machine so as to knit the breathable layer thereof into various thickness with upwards/downwards sustainability and rebound flexibility structure thereby.
